(19) United States
(12) Patent Application Publication
(10) Pub. No.: US 2025/0331973 A1
(43) Pub. Date: Oct. 30, 2025

(54) APPLICATION NOZZLE FOR CLEANING AN IMPLANT PART, IN PARTICULAR FOR USE IN A SYSTEM FOR CLEANING A COMPONENT CONTAMINATED WITH BIOFILM, IN PARTICULAR AN IMPLANT PART

(71) Applicant: GALVOSURGE DENTAL AG, Widnau (CH)

(72) Inventors: Holger ZIPPRICH, Seeheim-Jugenheim (DE); Erich FORSTER, Rüfenach (CH); Martin KAISER, Hassfurt (DE)

(21) Appl. No.: 18/866,623

(22) PCT Filed: May 19, 2023
(86) PCT No.: PCT/EP2023/063535

§ 371 Date: Nov. 18, 2024

(30) Foreign Application Priority Data
May 19, 2022 (DE) 102022205028.2
Aug. 3, 2022 (DE) 102022119524.4

(51) Int. Cl. A61C 19/06; A61C 17/02

(52) U.S. Cl. CPC A61C 19/063; A61C 17/02

(57) ABSTRACT

An application nozzle (20, 56, 56′, 56″) for applying a dental active substance in the oral cavity of a patient, in particular for a system (1, 1′) for cleaning an implant part (2) contaminated with biofilm, comprises according to the invention a base or nozzle body (30, 72) in which, on the one hand, at least one media channel (32, 76) and, on the other hand, a number of electrical conductor elements (34, 36, 84, 86) are integrated, the base or nozzle body (30, 72) being designed as a base or nozzle body (30, 72) which extends flat in a longitudinal direction from a connection region (26, 80) towards a free treatment end (28, 82) and tapers in its cross-section in the direction of the treatment end (28, 82).

The invention relates to an application nozzle for applying a dental agent in the oral cavity of a patient for cleaning an inserted dental implant. It also relates to a system for cleaning a component contaminated with biofilm, in particular an implant part, using such an application nozzle.

From WO 2014/075755 A1, WO 2014/122187 A1, WO 2014/122188 A1, WO 2016/023998 A1 and WO 2021/018871 A1, the disclosures of which are fully incorporated by reference, a treatment element, in particular for use with an implant part, and a method for cleaning a dental implant part are each known. Such cleaning of an implant part may be desirable or necessary to ensure the preservation of the inserted implant in the bone substance. A biofilm can form on the solid surface of implants, which is surrounded by tissue and tissue fluid and contains bacteria that can ultimately lead to chronic and recurring infections. This condition is known as peri-implantitis. Similar to periodontitis, a combination of neglected oral hygiene, adhesion of biofilm to the usually microrough surface of the dental implant and other factors is the cause of the full-blown peri-implantitis, which is characterized by increasing stress and destruction of the hard and soft tissue. The areas where the hard and/or soft tissue recedes are usually covered with a biofilm.

The cleaning process described in the above-mentioned publications is based on the concept of killing and removing the biofilm or germs forming the contamination starting from the implant surface without damaging the implant surface. For this purpose, an electrolytic process is provided in which ions (cations and/or anions) are transported through the biofilm by means of electrostatic forces. These ions react chemically or electrochemically on the implant surface. These reactions create new compounds and/or convert the ions themselves and/or parts of these ions into the atomic state. There is also the possibility that the ions react with the surface material (e.g. formation of an oxide layer or material removal). On the one hand, this process kills germs due to the chemical substances formed, but on the other hand it also causes the formation of gas bubbles that mechanically remove the biofilm. This effect, which provides the mechanical component of the mode of action in addition to the chemical one, can be the most important active component, especially when removing biofilm, as the biofilm can be comparatively resistant to chemical or biological effects due to its complex properties and the compound formed. However, the mechanical lifting effect by means of the gas bubbles formed breaks the bond, so that the components of the biofilm are also made accessible again for chemical or biological effects.

The germicidal effect of this process is based on various effects. Firstly, the application of an electrical voltage transports ions from the biofilm itself (including from the bacteria) to the anode or cathode. This can lead to the killing of bacteria and viruses. In addition, the ions can undergo biochemical reactions as they pass through the biofilm, which can also lead to the killing of bacteria and/or viruses. A further possibility of killing is that the new compounds formed on the implant surface have an antibacterial and/or antiviral and/or anti-fungal effect. This can of course also happen when the ions change to the atomic state.

The treatment element described in the aforementioned applications is specifically designed to carry out this cleaning process directly on the inserted dental implant, i.e. preferably while the abutment is in the bone in the patient's mouth. For this purpose, the treatment element is designed to be connected directly to the inserted abutment part and then to apply a suitable treatment liquid, which can serve as the basis for the desired electrolytic process when exposed to an electric current, in the immediate vicinity of the inserted abutment part in the affected area of the adjacent bone substance and to apply the electric current. To use this treatment element, it is therefore necessary to establish both mechanical and electrical contact with the inserted post part. For this purpose, in the design of the treatment element described in the aforementioned application, the prosthetics on the dental implant and possibly also its abutment must generally be temporarily removed for the purpose of fixing it to the abutment part. The performance of such a procedure is therefore primarily to be regarded as therapeutic in the sense that there is already an inflammation in the oral region in the vicinity of the implant and an existing biofilm must be detached and removed. The measures to be carried out during treatment are correspondingly far-reaching.

There is now also a desire or need to make the described treatment concept, i.e. the combined application of an electric current and an electrolytic cleaning fluid to the affected implant, suitable for preventive or prophylactic treatments. In contrast to therapeutic treatment, which can be associated with corresponding expense, the treatment concept should be designed to be particularly flexible and easy to use.

The invention is therefore based on the task of providing an application nozzle for a treatment system of the above-mentioned type, with which the application of the above-mentioned treatment concept is also made possible in prophylaxis in a particularly simple and cost-effective manner. Furthermore, a treatment system particularly suitable for the use of the application nozzle is to be specified.

With regard to the application nozzle, this object is achieved in accordance with the invention with a nozzle body in which, on the one hand, at least one media channel and, on the other hand, a number of electrical conductor elements are integrated for supplying a cleaning electrolyte from the connection region to the treatment end, the nozzle body being designed as a body which extends flat in a longitudinal direction from a connection region to a free treatment end and tapers in its cross-section in the direction of the treatment end.

Advantageous embodiments of the invention are the subject of the subclaims. Further and/or alternative advantageous embodiments of the invention, as well as further embodiments regarded as independent inventions, also result from the description of the figures.

The invention is based on the consideration that, on the one hand, particularly easy handling of the system components is desirable when using the aforementioned treatment concept in prophylaxis. On the other hand, in such an application, treatment should be possible without further intervention in the substance, for example temporary disassembly of the prosthetics or the like. According to the invention, it is therefore assumed that the treatment takes place as part of a regular dental examination or prophylaxis treatment, in which the inserted implant is to be treated and contacted in the area of its anchoring in the bone-without prior removal of the prosthetics or the like. Access to the implant surface to be treated should therefore essentially be via the tooth pockets associated with the implant, in which the causative bacteria would accumulate in any case in the event of an incipient inflammation. The application nozzle should therefore be designed in particular for good usability within the respective tooth pockets and/or between the implant and adjacent tooth with a correspondingly compact design.

In order to take this into account, according to one aspect of the invention, the design of the application nozzle or the treatment head is provided as an essentially flat component. This is not intended to mean that the treatment head could or should be two-dimensional; rather, it is to be understood here that the application nozzle or the nozzle body forming it should be a body which extends essentially along a basal plane or ground plane, but which nevertheless has a certain thickness in the third spatial direction. Seen in cross-section, however, this also means that the lateral expansion of the nozzle body in the basal plane is significantly greater than the thickness in the direction perpendicular to it. For the application, this means that the free or treatment end of this nozzle body can be inserted comparatively easily into the aforementioned tooth pockets, for example by aligning the basal plane of the nozzle body essentially parallel to the outer surface of the implant.

Furthermore, the cross-section of the nozzle body should taper towards the treatment end. The free or treatment end of the nozzle body therefore essentially has a flat, comparatively narrow or even tapered contour, so that it is particularly easy to insert into the tooth pockets.

According to one aspect of the invention, the conductor element or elements are positioned in or on one of the media channels in such a way that they are wetted by the cleaning electrolyte flowing in the respective media channel. This ensures reliable electrical contacting and thus the desired process control as described above.

In order to enable the application of comparatively large quantities of cleaning electrolyte into the tooth pocket with a particularly compact design, and in particular to be able to flood the pocket properly if required, the nozzle body should also be provided with a plurality of outflow openings in an outflow area arranged in the region of the treatment end, which can be used jointly for the application of the electrolyte. For this purpose, according to one aspect of the invention, a branching media channel is integrated into the nozzle body, which opens into a plurality of application openings on the outlet side, so that these can be supplied with electrolyte via a common supply. Alternatively, there can also be several media channels on the inlet side, which transport the same medium or are connected to the same media container or to different media containers, which all contain almost the same or the same medium.

In particular, the nozzle body can have a triangular contour when viewed from above.

In an advantageous further development, a number of the outflow openings are arranged in an outflow direction aligned laterally to the longitudinal axis. This makes it easy to flood the entire space in the tooth pocket around the free or treatment end of the nozzle body with cleaning electrolyte.

This allows the electrolyte to be applied specifically in the area of the free or treatment end of the treatment head or the application nozzle, in particular enabling precise application into the tooth pocket.

According to one aspect of the invention, the application nozzle can generally be used as a treatment head in a system for cleaning a component contaminated with biofilm or can be provided for this use.

In view of the intended use of the application nozzles in the dental care and prophylaxis sector, in order to enable the manufacture of an enormously large number of such application nozzles and thus mass production, a particularly suitable design is advantageously selected for this purpose, which allows the provision of a functionally reliable application concept even when using comparatively inexpensive materials. In order to take this into account, a foil material is advantageously provided as the base material for the manufacture of the nozzle body of the nozzle, which can be built up by laminating a plurality of foil layers on top of each other to form a suitable composite body. The base body or nozzle body, in which the media channels opening out on the outlet side in the respective application opening are arranged, is thus formed in this advantageous embodiment, which is regarded as independently inventive, by a layered body constructed as a laminate from a plurality of pieces of film.

The media transport channels provided for transporting the electrolyte to the intended delivery point in this composite body, which open into the application openings of the composite body on the output side, can be provided by recesses made in the respective film. The use of a film-based technology with subsequent lamination in particular enables enormous flexibility in the design and insertion of such media channels, as the free or empty spaces required for these within the composite body can be created in a variety of ways and geometries by suitable shaping in the respective film, for example by the particularly preferred punching or laser cutting. The media channels can be produced in particular by using lamination technology, for example by producing a central film with corresponding cut-outs and then laminating the upper and lower sides together with a continuous film to form a composite body in the form of a film sandwich. In this way, the media distribution in the individual channels can be very precisely controlled by their spatial design.

Advantageously, polyamide is provided as the base material for the films or film pieces; alternatively, however, another suitable film material such as PVC, PP or PE or even a combination of different film materials can be considered favorable. According to one aspect of the invention, the choice of material is made in particular with a view to ensuring that the application nozzle is also suitable for use by trained medical personnel, for example in connection with treatment by a dentist. Particularly preferably, the film material is selected with regard to its material properties, such as in particular its rigidity or strength, in such a way that the rigidity of the laminate or composite body formed from the film pieces is not too great and thus injuries in the oral cavity are largely excluded.

In a particularly advantageous embodiment, which is regarded as independently inventive, the nozzle body is constructed from at least three film layers which differ in their material properties and are functionally adapted to different specifications. In particular, according to one aspect of the invention, a central film layer arranged between two adjacent film layers can be formed entirely or partially from a harder film material, i.e. in particular with a different Shore hardness or modulus of elasticity, than the two adjacent film layers. The central film layer or one of the film pieces forming it can thus define the contour or spatial shape of the nozzle body in the manner of a support structure, whereas the comparatively softer outer film layers can be designed to be flexible and deformable and thus significantly reduce the risk of injury in the event of contact, for example, of the oral mucosa with the nozzle body.

According to one aspect of the invention, the media channels are formed by the respective recess in a central or inner film, which is laterally bounded by the respective side edges of the adjacent film pieces. The respective media channel is then bounded on the top and bottom by the correspondingly laminated, continuous base or cover film. In order to be able to safely and reliably provide comparatively large free cross-sections of the respective media channels suitable for the passage of even larger quantities of electrolyte with correspondingly wide recesses in the middle or central film, a number of the media channels are provided with integrated spacers in an advantageous embodiment which is regarded as independently inventive.

In a particularly advantageous embodiment, which is also regarded as independently inventive, the application nozzle is also designed with regard to its geometric configuration and dimensions for the intended use for the precise application of electrolytes into the oral region of a patient. In particular, it is advantageously taken into account that an application into the interdental spaces or the dental pockets of the patient could also be intended. According to one aspect of the invention, an application nozzle particularly suitable for this purpose has a nozzle body constructed as a laminate of the foil pieces with a total thickness of 0.3-2 mm, preferably of 0.5-1.5 mm, particularly preferably of 0.7-1.2 mm. Corresponding to this, the pieces of film forming the laminate advantageously each have a film thickness of 50-500 μm, preferably of 80-350 μm, particularly preferably of 100-250 μm.

In an advantageous further development, the application nozzle as described above is designed as a disposable or single-use product and is therefore intended for single use only.

According to one aspect of the invention, the application nozzle is provided with electrical conductor elements in order to reliably adjust the current flow intended for the aforementioned treatment concept. On the one hand, it is intended to establish the current flow via the cleaning electrolyte guided in the media channels so that the basic processes can be triggered. In order to ensure this, according to one aspect of the invention, a number of conductor elements assigned to a first electrical polarity are integrated into the nozzle body and positioned in or on one of the media channels in such a way that the respective conductor element is wetted by the cleaning electrolyte when it flows in the respective media channel. In the particularly preferred media-side parallel connection of two or more media channels, which can be connected on the inlet side via a branching point arranged in the nozzle body or also via a distribution system arranged on the flow side upstream of the nozzle body to a common electrolyte container or to a plurality of functionally interconnected electrolyte containers, such a conductor element assigned to the first polarity is preferably arranged in several, at least two, of these media channels. According to this aspect of the invention, at least two conductor elements assigned to the first electrical polarity are thus integrated into the nozzle body.

Furthermore, direct electrical contact with the implant is important for the intended mode of action during implant treatment. This should also take place via the application nozzle, since in the present case, unlike in the concepts described at the beginning, disassembly of the prosthetics is not provided for and thus electrical contacting of the implant via its inner region or the upwardly exposed region is not possible. In order to nevertheless enable reliable direct electrical contacting of the implant, according to one aspect of the invention, a conductor element associated with a second electrical polarity is integrated into the nozzle body, which, viewed in the longitudinal direction, projects beyond the treatment end formed by the nozzle body. This conductor element is thus exposed at the end and can therefore be brought into direct contact with an exposed outer surface of the implant, for example also within the tooth pocket, and thus into electrical contact. To a certain extent, this should form the electrical opposite pole to the conductor elements described above, so that the current flow can be adjusted by connecting a suitable current or voltage source.

In a particularly preferred embodiment, the treatment head is designed as a disposable or disposable product. This can be achieved, for example, by destroying the treatment head after use, i.e. after removal from the other components of the treatment system used, or rendering it unusable in some other way.

According to an aspect regarded as independently inventive, the application nozzle is used in a system for cleaning a component contaminated with biofilm, in particular an implant part.

With regard to the system for cleaning a component contaminated with biofilm, in particular an implant part, with such an application nozzle, the stated task is solved with a handle piece which is provided with a number of electrical and media connections in such a way that both the electrical conductor elements and the media channels of the application nozzle can be connected to corresponding electrical and media supply lines in the handle piece.

In a particularly advantageous embodiment, the treatment system is designed as a mobile device in which fixed connections to external peripheral devices are not required. For this purpose, a replaceable reservoir for cleaning electrolyte is advantageously arranged in the handle.

The advantages achieved with the invention consist in particular in the fact that the design of the nozzle body as a flat component tapering towards the free end means that the intended treatment of the inserted implant can be carried out via the associated pocket, to which access is made possible via the aforementioned spatial shape. Major preparatory measures for carrying out the treatment, such as removing the prosthetics, can therefore be omitted. The media channel integrated into the nozzle body and branching out there also makes it particularly easy to apply the electrolyte evenly and in a way that fills the entire space, in particular for reliable application to the entire tooth pocket, via the plurality of downstream application openings.

An embodiment of the invention is explained in more detail with reference to a drawing. It shows in:

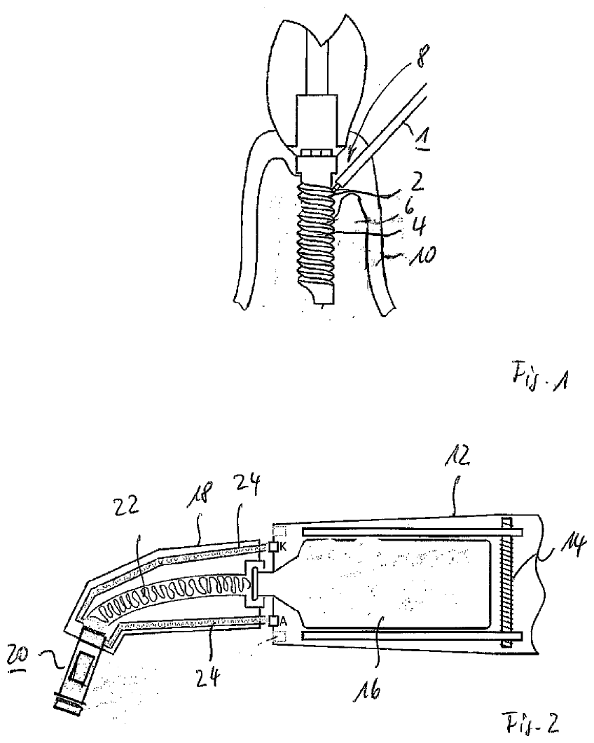

FIG. 1 a dental implant inserted into the oral bone of a patient,

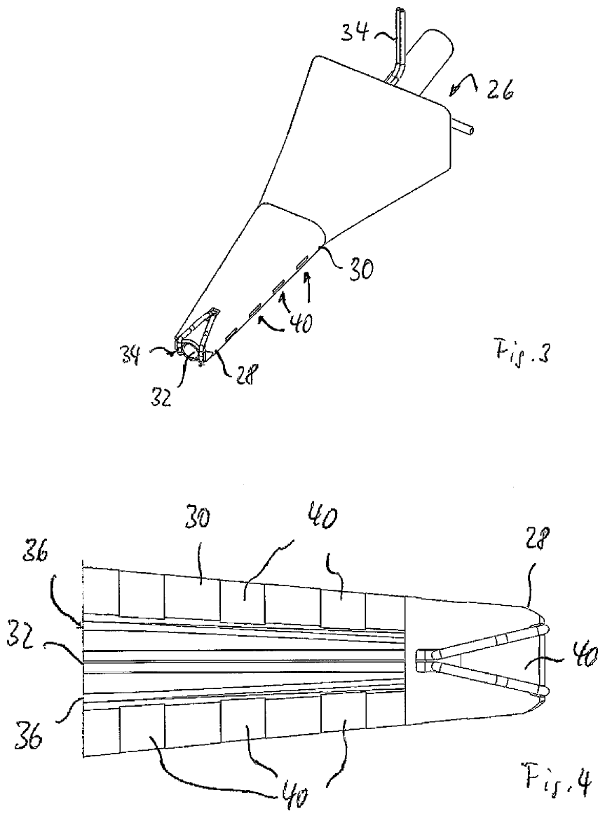

FIG. 2 a schematic of a mobile treatment system,

FIG. 3 a treatment head of the treatment system according to FIG. 2 in perspective view,

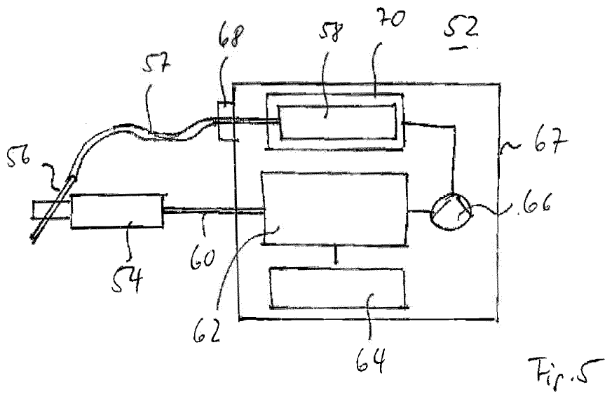

FIG. 4 the treatment head according to FIG. 3 in longitudinal section,

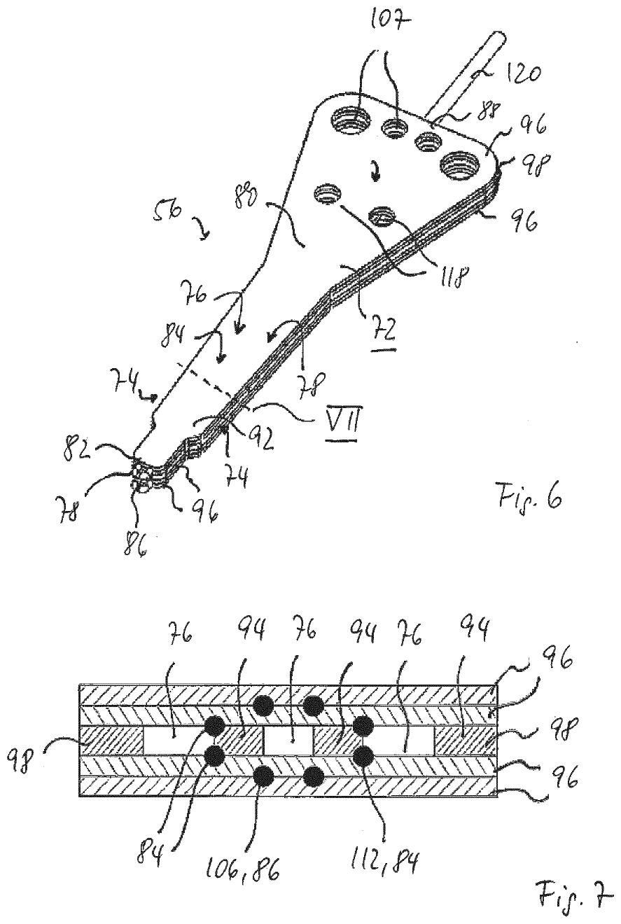

FIG. 5 a schematic of a stationary treatment system,

FIG. 6 a perspective view of an application nozzle of the treatment system according to FIG. 5,

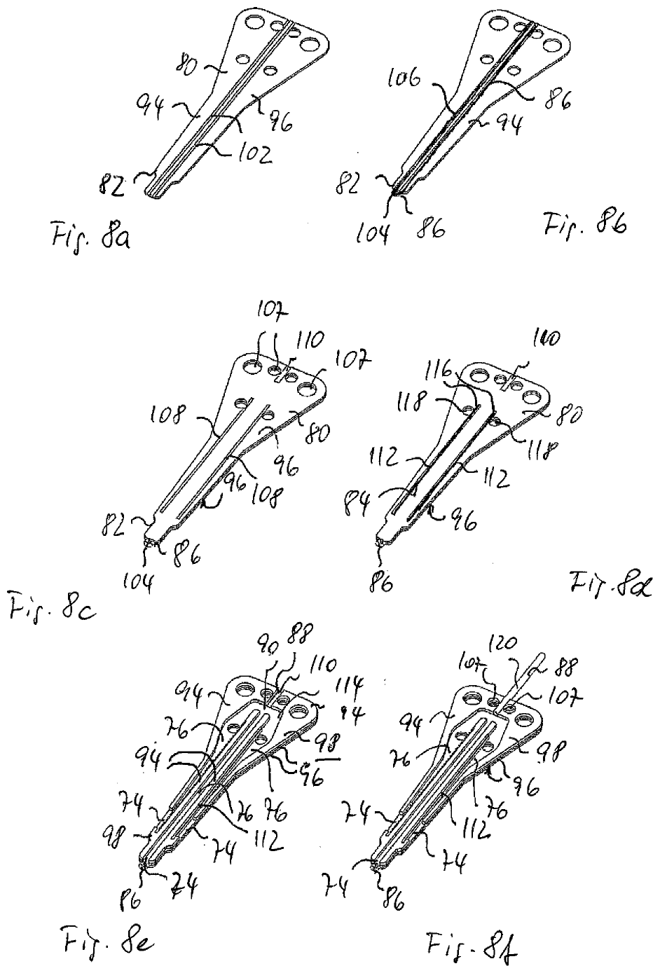

FIG. 7 a cross-section of the application nozzle according to FIG. 6,

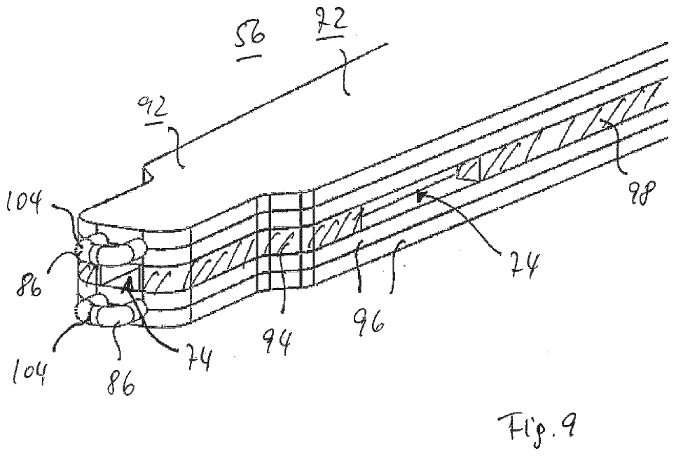

FIG. 8 a perspective view of a base segment of the application nozzle according to FIG. 6 in a sequence of manufacturing steps,

FIG. 9 a sectional enlargement of the treatment area of the active ingredient applicator according to FIG. 8,

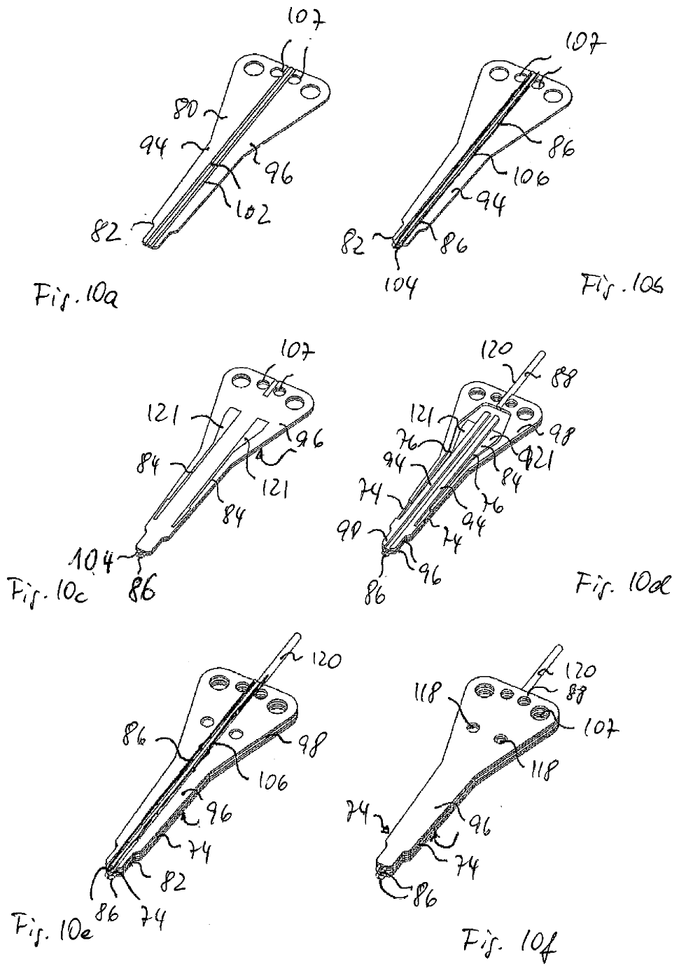

FIG. 10 a basal segment of the application nozzle according to FIG. 6 in an alternative design in a perspective view in a sequence of manufacturing steps,

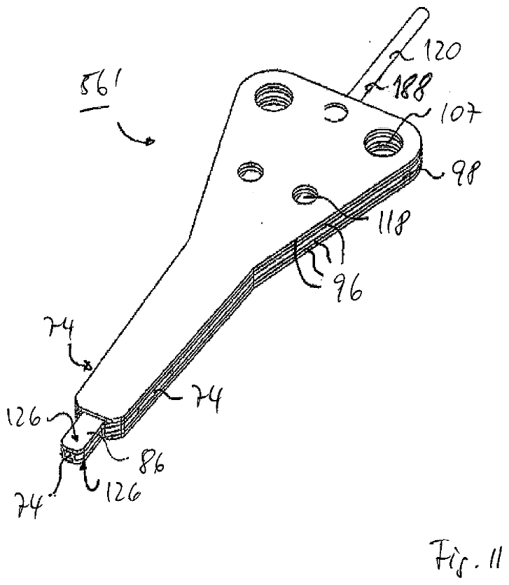

FIG. 11 a perspective view of an alternative embodiment of an application nozzle of the treatment system according to FIG. 5,

FIG. 12 a perspective view of a base segment of the application nozzle according to FIG. 11 in a sequence of manufacturing steps,

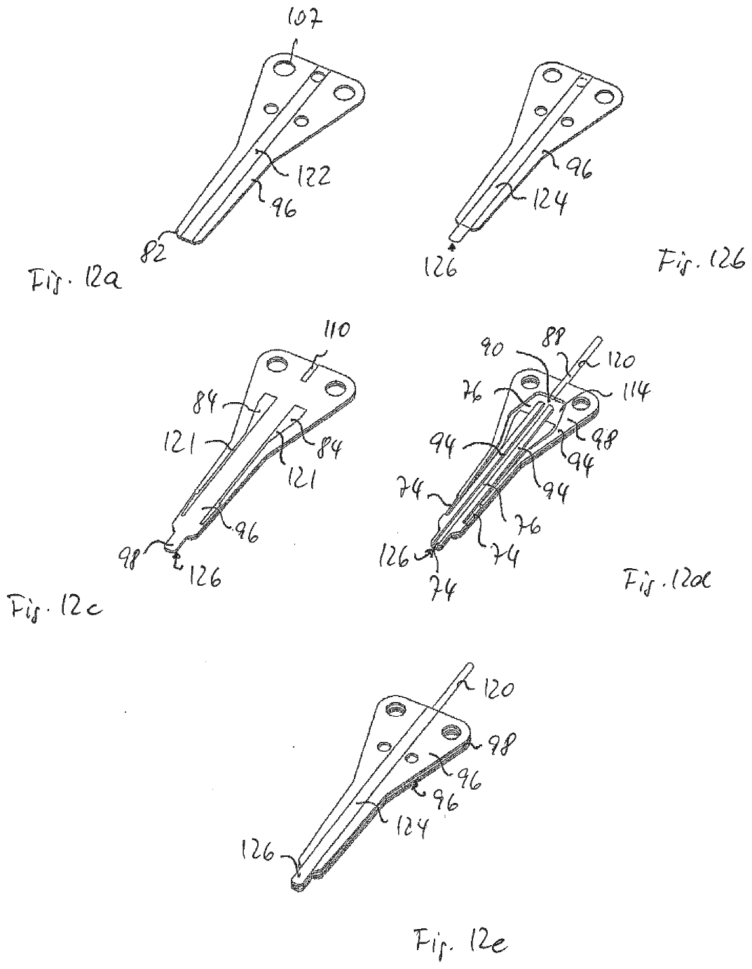

FIG. 13 a further alternative embodiment of an application nozzle of the treatment system according to FIG. 5 in plan view,

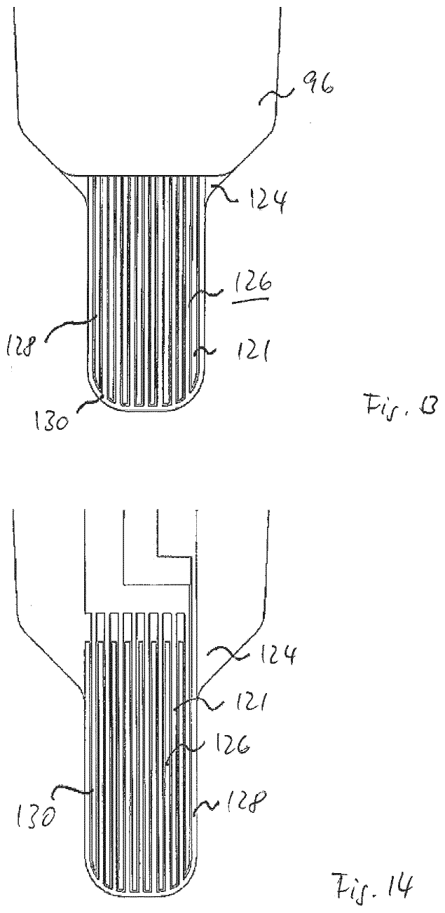

FIG. 14 a top view of an electrode area of the application nozzle according to FIG. 13,

FIG. 15 a further alternative embodiment of an application nozzle of the treatment system according to FIG. 5 in perspective view,

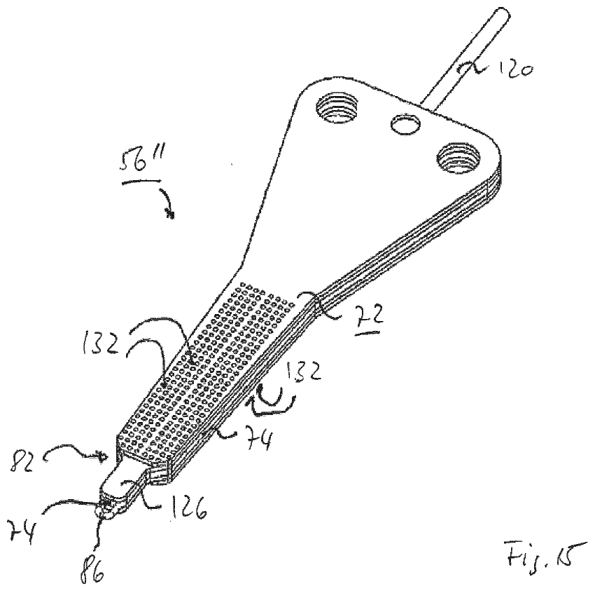

FIG. 16 another alternative embodiment of an application nozzle of the treatment system according to FIG. 2 or 5 in a sequence of manufacturing steps,

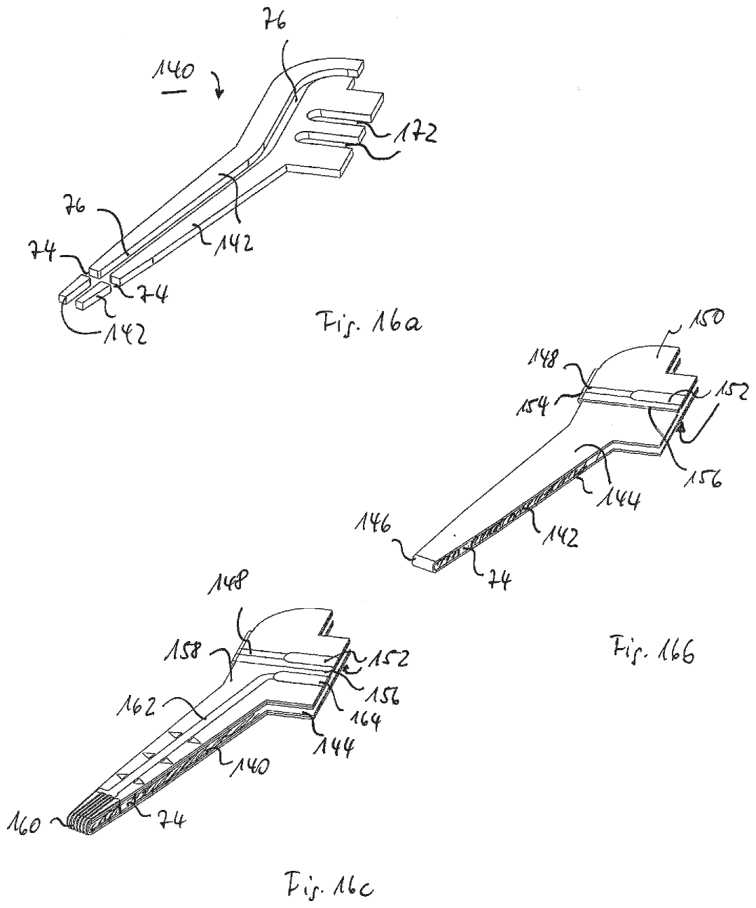

FIG. 17 a magnified view of a treatment end of the application nozzle according to FIG. 16,

FIG. 18 in enlarged view the contact head of the application nozzle according to FIG. 16,

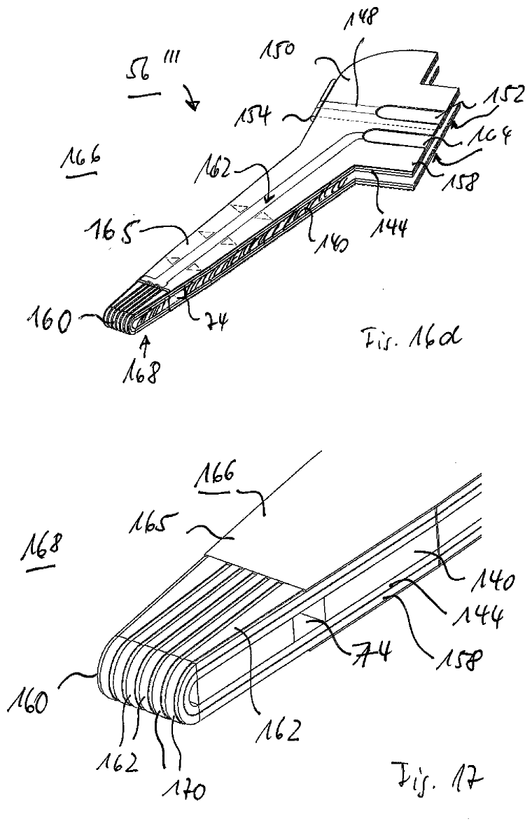

FIG. 19 a treatment head comprising the application nozzle according to FIG. 16 in partial section,

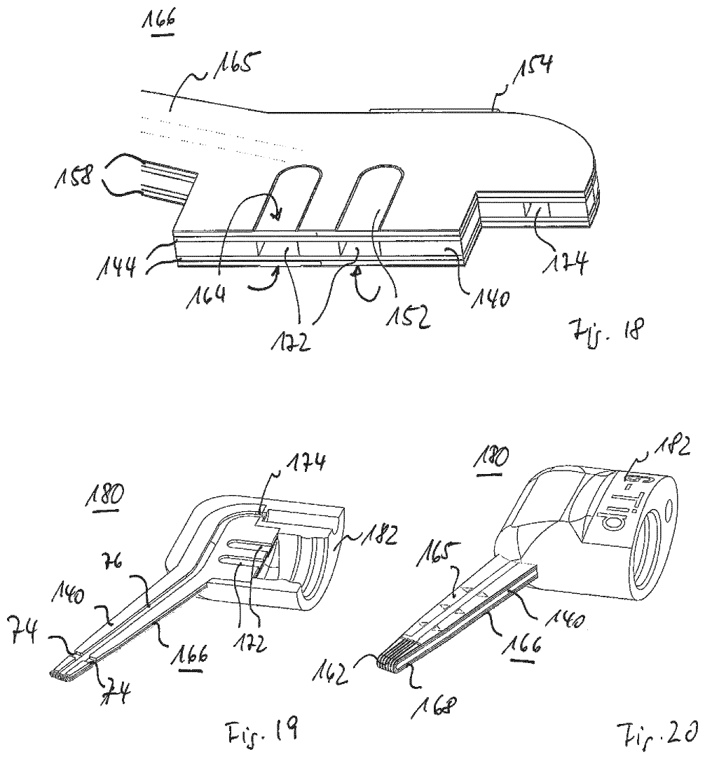

FIG. 20 the treatment head according to FIG. 19 in lateral view,

FIG. 21 in enlarged view an alternative embodiment of the treatment end of the application nozzle according to FIG. 16,

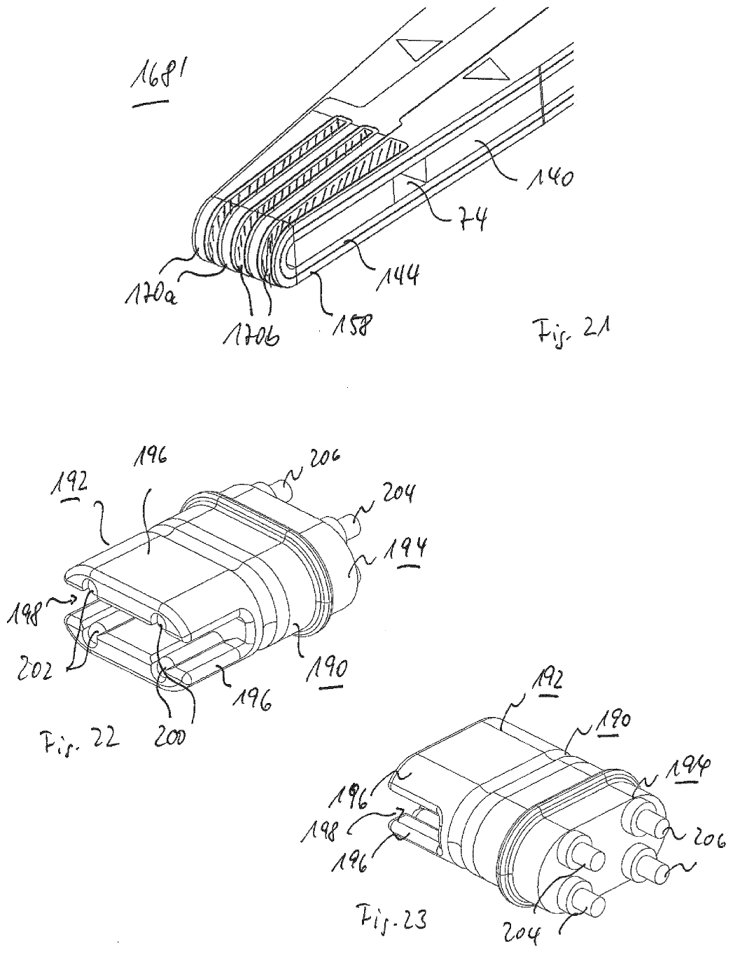

FIG. 22 a contact plug for the treatment head according to FIG. 19 in perspective front view, and

FIG. 23 a perspective rear view of the contact plug according to FIG. 22.

Identical parts are marked with the same reference signs in all figures.

In general, the problem with dental implant systems, especially with two-piece implant systems, is that inflammation or foci of inflammation can occur if bacteria or germs penetrate into the tissue area near the insertion site, especially in the area of the external thread inserted into the jaw. Such inflammations, especially those resulting from so-called peri-implantitis, can lead to serious damage to the tissue and bone in the area of the insertion site, especially if they develop and consolidate over a longer period of time. Without suitable countermeasures, these impairments can lead to the entire implant system having to be removed from the bone and, after bone augmentation, fitted with a new implant system or replaced with other prosthetics. This extremely undesirable effect caused by peri-implantitis can therefore lead to a total loss of the implant system, so that further surgical measures such as scraping out the affected area of the jawbone and re-fitting with an implant system may be necessary. Such removal can also result in bone loss or other loss of tissue substance, which in extreme cases can lead to a situation where a new restoration with another implant is no longer possible. Such a need for a new restoration caused by peri-implantitis can also occur after comparatively long periods of time after the first insertion of the implant system, for example up to several years or even decades.

The germs or bacteria observed in connection with peri-implantitis can in principle colonize the interior of the implant components, but generally prefer to adhere directly to the surface of the dental implant inserted into the jawbone in the area of contact with the surrounding tissue or bone material, i.e. in particular in the area of the external thread. In this area, the surface of the dental implant can be provided with a roughening or the like in order to particularly favor ingrowth into the tissue or bone and to support the healing of the dental implant after insertion. However, it is precisely in the area of such a roughened surface, which is actually considered to be particularly favorable for the implant system, that germs or bacteria can increasingly colonize, whereby the roughness makes it even more difficult to specifically remove the existing germs or bacteria.

There is therefore an urgent need for suitable countermeasures to be able to effectively combat the focus of inflammation and kill and/or remove the invading germs in the event of imminent or existing peri-implantitis while preserving the implant system already in place, so that healthy tissue or healthy bone substance can then form again in the area around the external thread. In addition to targeted killing of the germs or bacteria in the affected area, it is also desirable to reliably remove their material residues and fragments from the affected area so that the affected area can then be filled with healthy tissue or bone material again and an intimate connection can form again between the outer surface of the dental implant and the surrounding tissue or bone material. In addition, the biofilm formed by the bacterial coating, including the organic residues of dead bacteria, should be reliably removed.

In order to make this possible, a treatment concept for cleaning a component contaminated with biofilm, in particular an implant part, is known from publications WO 2014/075755 A1, WO 2014/122187 A1, WO 2014/122188 A1, WO 2016/023998 A1 and WO 2021/018871 A1, the disclosures of which are incorporated in full (“in-corporation by reference”), in which the contaminated surface of the implant is wetted with an electrolytic cleaning fluid and subjected to a current flow. The combination of a suitably selected electrolyte and current flow causes, among other things, gas bubbles to form directly on the implant surface, which blast off any adhering biofilm and thus help to clean the surface. A continuous supply of electrolyte rinses the area to be cleaned (e.g. the pocket) and thus removes the dissolved concrements.

In contrast to the publications mentioned above, however, the present case is not primarily intended and aimed at a therapeutic treatment of peri-implantitis or tissue inflammation that has already occurred, but rather a prophylactic treatment in which the development of inflammation or its further expansion and spread is to be prevented as part of a standardized, precautionary treatment. This can, for example, prevent existing mucositis from developing into peri-implantitis.

The use of a treatment system 1 provided for this purpose is shown schematically in FIG. 1. This shows a dental implant 2 installed in the oral bone of a patient. For clarification, FIG. 1 shows a so-called tooth pocket 8 adjacent to the dental implant 2 in the region of its external thread 4 and the jawbone 6, which usually forms in the manner of an increasingly opening gap between the tooth substance or the jawbone 6 and the surrounding soft tissue 10. Bacteria preferentially accumulate in such a pocket 8, which can lead to subsequent inflammation, in the case of healthy teeth in the form of periodontitis and in the case of an inserted dental implant 2 to the aforementioned peri-implantitis.

Treatment system 1 is intended to counteract this in the form of a prophylaxis and already at an early stage, i.e. when bacterial infestation or biofilm is beginning to form or is already spreading. In terms of its mode of action, this is designed according to the concept of the above-mentioned publications: On the one hand, it is designed to specifically kill the germs or bacteria present in the insertion area of the dental implant 2 by selectively supplying a bacteriocidal cleaning agent or disinfectant that is compatible with the human organism. On the other hand, it is designed to detach any residues or fragments of germs and/or bacteria already adhering to the surface of the dental implant 2, particularly in the area of the external thread 4, from the outer surface of the dental implant 2 by applying a suitable current or current surges, so that they can then be washed out.

The treatment system 1 is designed as a mobile system in a first embodiment, which is independently regarded as inventive both with regard to the design of the system and with regard to the intended method steps of the treatment method, as shown schematically in FIG. 2. The system 1 comprises a handle 12, which is provided with suitable reservoirs and storage elements with regard to the intended mode of operation, i.e. the application of electrical current pulses to the dental implant 2 in the region of the tooth pocket 8 as well as with a suitably selected electrolytic cleaning fluid. For this purpose, a suitable battery 14 (or any other suitable current or voltage source) on the one hand and a reservoir 16 for cleaning electrolyte on the other hand are integrated into the handle 12. The storage container 16 is designed as a replaceable storage container 16 so that it can be easily refilled after the contents have been used up. In particular, the storage container 16 could be designed in the form of an ampoule for a medical agent, whereby the media-side connection can be implemented via a Luer connection using established filling and connection concepts.

In the example shown, the handle 12 is connected on the media side and also electrically to a transition piece 18. These components, which could in principle also be designed as a single functional part, form a reusable component that can in principle be used in a large number of treatments, for example as part of a standardized preventive measure for a large number of patients. To enable the actual treatment, the actual application nozzle, referred to as treatment head 20 in this design example, is connected to it. In view of hygiene and care considerations, the treatment head 20 is designed for single use only and is therefore a disposable product. The treatment head 20 is provided with a number of electrical and media connections in such a way that both the electrical conductor elements and the media channels of the treatment head 20 can be connected to corresponding electrical and media supply lines 22, 24 in the handle 12 and in the transition piece 18.

The treatment head 20, also referred to as a nozzle, which is shown in FIG. 3 in perspective view and in FIG. 4 in longitudinal section, has a base body 30 extending in a longitudinal direction from a connection side 26 towards a free treatment end 28, in which a number of media channels 32 for supplying the cleaning electrolyte from the connection side 26 towards the treatment end 28 and a number of electrical conductor elements 34, 36 are integrated. It is specifically designed in a particularly simple manner for the intended use in a prophylactic treatment, i.e. the electrical contacting of the dental implant 2 and the targeted application of the cleaning electrolyte into the tooth pocket 8, in a manner which is regarded as independently inventive. Particular account is taken of the fact that the practitioner should contact the implant 2 precisely despite the very confined space. To make this possible, the spatial shape of the base body 30 is suitably selected taking into account the fact that a tooth pocket 8 is usually formed in the form of a gap extending along the tooth or implant surface.

In order to take this into account, according to one aspect of the invention, the treatment head 20 is designed as an essentially flat component in the form of a flat spatial body. The treatment head 20 or the base body 30 forming it is thus designed as a body extending essentially along a basal plane or ground plane, the thickness of which, viewed in cross-section, is kept significantly smaller than its lateral extension in the basal plane. For the application, this means that the free or treatment end 28 of the base body 30 can be inserted comparatively easily into the tooth sockets 8, for example by aligning the basal plane of the base body 30 essentially parallel to the outer surface of the implant 2.

Furthermore, the base body 30 tapers in its cross-section in the direction of the treatment end 28. The free or treatment end 28 of the base body 30 thus essentially has a flat, comparatively narrow or even tapered contour, so that insertion into the tooth pockets 8 is particularly easy. In the embodiment shown, this results, for example, as can be seen from the representation in longitudinal section according to FIG. 4, in the fact that the base body 30 has a contour shaped in the manner of a triangle in planar view.

In order to reliably ensure the conceptually intended direct electrical contacting of the dental implant 2 as part of a prophylaxis treatment, the base body 30 has an integrated conductor element 34 assigned to a first electrical polarity, which protrudes beyond the treatment end 28 formed by the base body 30 when viewed in the longitudinal direction. At the end, this conductor element 34 is thus exposed and can therefore be brought into direct contact with an exposed outer surface of the implant 2 within the tooth pocket 8 and thus into electrical contact. It is preferable to switch the implant 2 cathodically; accordingly, the conductor element 34 is provided for switching in cathodic polarity.

Furthermore, to complete the electrical current path provided within the framework of the treatment concept, at least two conductor elements 36, in the embodiment example exactly two, assigned to a common second electrical polarity are integrated into the base body 30. These form the electrical opposite pole to the conductor element 34 described above and are accordingly provided in the embodiment example for an anodic circuit. The current flow can thus be adjusted by connecting the current or voltage source provided in the handle 12. The conductor elements assigned to the second electrical polarity serve, in accordance with the concept described in the above-mentioned publications, to establish the current flow via the supplied cleaning electrolyte, so that the basic processes can be triggered.

In order to ensure this, the treatment head 20 is designed for precise application of the cleaning electrolyte into the tooth pocket 8 so that the desired current path can be created via it. For this purpose, the base body 30 is provided with a number of outflow openings 40 for the cleaning electrolyte, each connected to one of the media channels 32, in an outflow area 38 arranged in the area of the treatment end 28. This allows the electrolyte to be discharged specifically in the area of the treatment end 28 of the treatment head 20 and thus, if necessary, directly into the respective tooth pocket 8.

In the present case, it is considered particularly important that the cleaning electrolyte is reliably applied in direct electrical contact with the anodically connected conductor elements 36. This ensures that—as intended by the concept—the current flow takes place via the electrically conductive cleaning electrolyte, which ultimately results in the desired generation of the ionic reactions and possibly the gas bubbles on the surface of the implant 2. In order to ensure this, the conductor elements 36 assigned to the anodic, second electrical polarity are positioned in or on one of the media channels 32 in such a way that they are wetted by the cleaning electrolyte when it flows in the respective media channel 32.

A number of the outflow openings 40 are also arranged in an outflow direction aligned laterally to the longitudinal axis, as can be seen, for example, in the perspective view in FIG. 3. This makes it easy to flood the entire spatial area in the tooth pocket 8 around the treatment end of the base body 30 with cleaning electrolyte.

In an alternative embodiment, the treatment system 1′ can also be designed as a stationary system, as shown in the embodiment example according to FIG. 5. This can be intended in particular for use in the context of a dental treatment, such as a cleaning or prophylaxis measure or also a therapeutic treatment. The treatment system 1′ comprises a central supply unit 52, to which an application nozzle 56 is connected via an intermediate handle or handpiece 54 as the actual treatment head or actual treatment element, which is designed for a single use only and thus as a disposable product with regard to hygiene and care considerations.

Even if the treatment head 20 described above in combination with the mobile treatment system 1 and the application nozzle 56 described in more detail below in combination with the stationary treatment system 1′ are explained, it goes without saying that in the context of the present both could also be used in the respective other treatment system 1, 1′ with otherwise identical design, i.e. the treatment head 20 described above instead of the application nozzle 56 in the stationary treatment system 1′ and the application nozzle 56 instead of the treatment head in the mobile treatment system 1.

The application nozzle 56 is connected to an electrolyte cartridge or ampoule 58 arranged in the supply unit 52 via connecting elements, in the embodiment example via PVC or silicone hoses 57, for connection on the media side. Furthermore, it is connected to a control unit 62 arranged in the supply unit 52 via electrical connection lines 60. The electrolyte cartridge 58 and the control unit 62 are arranged in a common outer housing 67 of the supply unit 52 together with a backup battery 64 provided for supplying power to the control unit 62 as required and with a pump 66. The application nozzle 56 is provided with a number of electrical and media connections in such a way that both the electrical conductor elements 60 and the media-side connecting hose 57 formed by the PVC/silicone hoses can be suitably connected.

The electrolyte ampoule 58 is intended to provide a cleaning electrolyte as disclosed, for example, in the publications WO 2014/075755 A1, WO 2014/122187 A1, WO 2014/122188 A1, WO 2016/023998 A1 and WO 2021/018871 A1, the disclosures of which are incorporated in full (“incorporation by reference”). The connecting hose 57 connecting the electrolyte ampoule 58 to the application nozzle 56 on the media side can be shut off via a hose valve 68 arranged in the area of its passage through the outer housing 67 and controllable via the control unit 62, which can start the electrolyte flow when it is opened and can stop it again when it is closed. In accordance with one aspect of the invention, the hose valve 68 can be designed in particular as a pinch valve which, in the shut-off mode for shutting off, deforms the comparatively soft hose material by squeezing it until it is completely shut off.

The electrolyte ampoule 58 is designed as a disposable product and thus as a replaceable storage container, so that it can be disposed of after the contents have been used up and replaced by a new ampoule. In particular, the electrolyte ampoule 58 could be designed in the form of an ampoule for a medical active ingredient, whereby the media-side connection can be designed via a Luer connection using established filling and connection concepts. In the embodiment shown, the electrolyte ampoule 58 is designed with a comparatively soft ampoule body in an embodiment considered to be independently inventive, which can be compressed by the user, for example during manual use, in order to dispense the active ingredient contained therein. According to a further aspect considered to be independently inventive, a pressure chamber 70 is positioned in the interior of the outer housing 67. The pressure chamber 70 is connected to the pump 66, which is designed as an air pump. As soon as the internal pressure in the pressure chamber 70 is increased sufficiently via the pump 66, this excess pressure, utilizing the deformability of the ampoule body, results in the ampoule body being compressed and the active ingredient contained therein being dispensed. In this independently inventive embodiment, an “encapsulated” application of the active ingredient is thus made possible, in which no direct contact with the ampoule body is necessary. Especially in combination with the pinch valve described above, access and process control from the outside, via electrical signal lines, and thus automation is possible in a particularly simple way.

The electrolyte ampoule 58 is thus placed in the pressure chamber 70 for operation of the system 1 according to this aspect of the invention. An overpressure of 0.8-1.5 bar, preferably 1.0-1.2 bar, is built up in the pressure chamber 70 via the pump 66 and kept as constant as possible. The resulting outflow of electrolyte is controlled by the hose valve 68. When open, the hose valve 68 enables the flow and stops it by squeezing the hose. The media flow from the electrolyte ampoule 58 can be suitably controlled and, if necessary, regulated by the interaction and suitable actuation of the pump 66 on the one hand and the hose valve 68 on the other.

Furthermore, the electrolyte ampoule 58 can also be pressed out using other mechanical, pneumatic or hydraulic systems.

With regard to the intended preferred use in the field of dental care or prophylaxis, the application nozzle 56 shown in FIG. 6 in perspective view is specifically designed for high functionality with a particularly simple design, so that production is also possible in enormously high quantities with only limited manufacturing costs. For this purpose, the application nozzle 56 has a nozzle body 72 as an essential functional component, in which a number of media channels 76 connected to application openings 74 on the outlet side for the active substance to be applied, i.e. in particular the cleaning electrolyte, and a number of electrical conductor elements 78 for generating the intended current flow through the cleaning electrolyte are integrated. It is specifically designed in a particularly simple manner for the intended use in a prophylactic treatment, i.e. the electrical contacting of the dental implant 2 and the targeted application of the cleaning electrolyte into the pocket 8, in a manner which is regarded as independently inventive. Particular account is taken of the fact that the practitioner should be able to contact the implant 2 precisely despite the very confined space. In order to make this possible, the spatial shape of the nozzle body 72 is suitably selected taking into account the fact that a pocket 8 is usually formed in the form of a gap extending along the implant surface.

In order to take this into account, according to one aspect of the invention, the design of the application nozzle 56 is provided in the manner of a spatial body held flat as an essentially flat component extending flat in a longitudinal direction from a connection region 80 to a free treatment end 82. The application nozzle 56 or the nozzle body 72 forming the same is thus designed as a body extending essentially along a basal plane or ground plane, the thickness of which, viewed in cross-section, is kept significantly smaller than its lateral extension in the basal plane. For the application, this means that the free or treatment end 82 of the nozzle body 72 can be inserted into the tooth pockets 8 comparatively easily, for example by aligning the basal plane of the nozzle body 72 essentially parallel to the outer surface of the implant 2.

Furthermore, the nozzle body 72 tapers in its cross-section in the direction of the treatment end 82. The free or treatment end 82 of the nozzle body 72 thus essentially has a flat, comparatively narrow or even tapered contour, so that insertion into the tooth pockets 8 is particularly easy. In the embodiment example, this results in the nozzle body 72 having a contour shaped in the manner of a triangle, at least in sections, when viewed from above.

In order to reliably ensure the conceptually intended direct electrical contacting of the dental implant 2 in the context of a prophylaxis treatment, at least two, in the embodiment example exactly two, conductor elements 84 assigned to a common first electrical polarity are integrated into the nozzle body 72. Furthermore, to complete the electrical current path provided within the framework of the treatment concept, the nozzle body 72 has, as a further one of the conductor elements 78, an integrated conductor element 86 assigned to a second electrical polarity, which, viewed in the longitudinal direction, projects beyond the treatment end 82 formed by the nozzle body 72. In the embodiment example shown in FIG. 6, even two such conductor elements 86 are provided. On the end side, these conductor elements 86 are thus exposed and can therefore be brought into direct contact with an exposed outer surface of the implant 2 within the pocket 8 and thus into electrical contact. It is preferable to switch the implant 2 cathodically; accordingly, the conductor elements 86 are provided for switching in cathodic polarity.

The conductor elements 84 are only indicated in FIG. 6. These form the electrical opposite pole to the conductor elements 86 described above and are accordingly provided in the embodiment example for an anodic circuit. The current flow can thus be adjusted by connecting the current or voltage source provided in the handle 54. The conductor elements 84 assigned to the first electrical polarity serve, in accordance with the concept described in the above-mentioned printed matter, to establish the current flow via the supplied cleaning electrolyte, so that the basic processes can be triggered.

To ensure this, the application nozzle 56 is designed for a precise and comprehensive application of the cleaning electrolyte into the pocket 8, so that it can be flooded as efficiently as possible and thus the desired flow path can be reliably produced via the cleaning electrolyte. For this purpose, the media channel 76 integrated in the nozzle body 72 and provided for supplying the cleaning electrolyte is branched in the sense that the media channel 76 starting from a media connection 88 splits into a plurality of channels at a branching point 90 within the nozzle body 72 and is connected via these on the outlet side to a plurality of the outflow or application openings 74 arranged in an outflow region 92 provided in the region of the treatment end 82. In the embodiment example, the application openings 74 connected in parallel on the media side in this way are positioned on both sides and with a lateral outflow direction at the treatment end 82, so that a uniform discharge of the cleaning electrolyte to both sides of the application nozzle 56 is possible. This allows the electrolyte to be discharged in a targeted manner into the complete spatial vicinity of the treatment end 82 of the application nozzle 56 and thus, if necessary, directly into the respective pocket 8. Specifically, it is thus possible to flood the pocket from apically via the treatment end 82 and laterally somewhat further up, in each case via the outflow or application openings 74.

With regard to the desired low-cost design suitable for large quantities, according to one aspect of the invention, the nozzle body 72, as can be seen particularly clearly in the cross-sectional representation according to FIG. 7, is designed in the manner of a laminate body as a layered body constructed from a plurality of pieces of film 94. The respective media channels 76 are formed in a film layer 96 of the laminate by a recess made in the respective laminated film. Due to this design of the application nozzle 56 or its nozzle body 72 as a film composite body or laminate, suitable application nozzles 56 can be provided inexpensively and in large quantities by comparatively simple means and with enormous flexibility in the spatial design.

The application nozzle 56 or its nozzle body 22 is formed in the manner of a laminate or stack of layers by a number of pieces of film 94 arranged one above the other and bonded, welded or otherwise connected to one another at their contact surfaces. The pieces of film 94 each have a film thickness d of approximately 100-250 μm and thus within a preferred range of 50-500 μm. The application nozzle 56 or its nozzle body 72 constructed as a laminate of the pieces of film 94 thus has a total thickness D of approximately 0.7-1.2 mm, i.e. within a preferred range of 0.3-2 mm, so that the desired insertion into the pocket 8 is possible without difficulty.

The media channels 76 can be created by punching or laser cutting into the respective piece of film 94, so that the application nozzle 56 can be designed with particularly simple means and with particular flexibility with regard to its spatial configuration and the type and number of media channels. As can also be seen from the representation in FIG. 4, a number of the media channels 76 can be provided with integrated spacers 100, so that a comparatively flat, comparatively wide media channel 76 can be formed to provide comparatively large flow cross-sections.

According to one aspect of the invention, the one-piece application nozzle 56 shown in FIG. 6 has this construction as a film layer or composite body or as a laminate for the entire nozzle body 72 forming the application nozzle 56. In the embodiment example, the application nozzle is also constructed from film layers 96, 98 which differ in terms of their material selection and parameters, a central middle film layer 98 of a first film material being covered on both sides by a respective side or outer film layer 96 of another film material. The film layer 98 and the film layers 96 differ in their material properties and are functionally adapted to different specifications. In the embodiment example, the central film layer 98 consists of a comparatively harder film material, i.e. in particular with comparatively greater Shore hardness or modulus of elasticity, whereas the other film layers 96 tend to be softer. The central film layer 98 can thus define the contour or spatial shape of the nozzle body 72 in the manner of a support structure, whereas the comparatively softer outer film layers 96 can be designed to be flexible and deformable and thus significantly reduce the risk of injury in the event of contact, for example, of the oral mucosa with the nozzle body 72.

In the embodiment shown, the application nozzle 56 consists in its entirety of such a film composite. This construction method becomes clear from the representation of the layer-by-layer sequence of the structure in FIG. 8 and its enlargement in FIG. 9.

The structure of the film layer package is shown in FIG. 5 by means of a sequence, starting from the first, lowermost film layer 96, with stepwise addition of the further film layers 96, 98. Accordingly, FIG. 8a shows the lowermost or first film layer 96, which is already adapted to the desired shape of the nozzle body 72 when viewed from above. Starting from the connection area 80, the width tapers in the direction of the treatment end 82. The piece of film 94, which has already been pre-cut in its outer contour in this way, is also provided with embossed grooves 102. In a subsequent step during the construction of the film stack, as shown in FIG. 8b, a conductor wire 106 bent at its free end 104 is inserted into these grooves to form the said conductor element 86. The bent end 104 thereby protrudes forwardly beyond the base surface formed by the piece of film 94, i.e. beyond the treatment end 82, as is intended for the conductor element 86 according to the design.

A further piece of film 94, also forming a film layer 96, is then applied to the lower film layer 96 provided in this way with the lead wire 106 and laminated on, for example. This resulting film stack is shown in FIG. 8c. The foil layer applied last covers the previously inserted conductor wire 106, so that the conductor element 86 formed by it is visible in this illustration only at the end 104 still protruding at the front beyond the treatment end 82. Thus, the conductor element at the end 104 serves to contact the implant and is insulated in the remaining area. Only via contact openings 107 provided throughout in all foil layers 96, 98 can the lead wire 106 thus still be electrically contacted, for example from the control unit. On its upper surface, the upper film layer 96 is also provided with embossed grooves 108. Furthermore, it comprises an embossed or stamped receiving groove 110 in the connection area 80.

A conductor wire 112 is then inserted into each of the grooves 108, as shown in FIG. 8d. The conductor wires 112 are used to form the above-mentioned conductor elements 84 associated with a first polarity. After the conductor wires 112 have been attached, the next film layer 98 is applied to the resulting layer stack, as shown in FIG. 8e. The foil layer 98 forms the central foil layer 98 and is made of a comparatively harder foil material, i.e. in particular with a comparatively greater Shore hardness or modulus of elasticity, the other foil layers 96 being rather softer. The film layer 98 can thus assume the function of a supporting or shaping layer, which gives the entire package a certain rigidity and mechanical stability.

It is clearly recognizable in FIG. 8e that the film layer 98 is made in several parts and is formed by a number of film pieces 94. The foil pieces 94 are arranged at a distance from each other, leaving openings 114 between them. These openings 114 form the media channels 76 integrated in the nozzle body 72, which can be designed with a large degree of freedom due to the possibilities for processing the foils (lasers, punching). In the embodiment example according to FIG. 8e, it can be clearly seen that, starting from the media connection 88 provided, the media channel 76 branches out at a branching point 90 provided in the connection area 80 and continues in three subsequent channel sections, one central and two lateral, up to the respective outflow openings 74. The common media connection 88 is thus connected to three outflow or application openings 74 via the media duct 76 branched in this way; these are thus connected in parallel on the media side.

The conductor elements 112 are also positioned in the two lateral of these three parallel-connected channel sections in such a way that they are wetted by the medium flowing in the respective media channel 76. This allows electrical contact to be established with the medium flowing there via the conductor elements 112. For contacting from the periphery, the conductor elements 112 are also, as can be seen in FIG. 8d, guided segment by segment at their end segments 116 through contacting holes 118 provided there. According to one aspect of the invention, these are arranged continuously through the entire film stack, so that, for example, a connecting plug could be inserted to establish an electrical contact with the respective conductor element 112.

This contacting concept using the contacting holes 107, 118 passing through the entire foil stack is also regarded as independently inventive. The fact that the conductor wires 106 integrated in the foil stack and running parallel to its basal plane can be used to achieve an overall flat design with only a low overall height. Reliable electrical contacting can nevertheless be achieved by inserting suitable connecting elements, such as connecting pins, into the respective contacting holes 107, 118 in a close-fitting manner and thus bringing them into intimate surface contact with the conductor segments running there without damaging them.

In particular, a connecting pin with a diameter slightly larger than the respective contacting hole 107, 118 can be used. When this is pushed into the respective contacting hole 107, 118, the latter deforms due to its geometry, so that intimate contact is made with the connecting pin. As described above, the conductor element can be designed as a wire, but preferably also as a foil or other flat element, since the deformability provides a particularly useful contacting option.

Alternatively, it is also possible to simply press a contact pin or the like onto a wire or a conductor track for contacting without the need for an opening or hole in the conductor track.

In a further step, a connecting tube 120 is inserted into the receiving groove 110 of the lower film layers 96, which is also continued in the film layer 98 by a corresponding recess 114 between two film pieces 94, to form the media connection 88, as shown in FIG. 8f. This can be formed as a metal tube or ceramic tube, or alternatively as a plastic tube or also as a rolled piece of foil. A tapered foil stack would also be possible. This would be a particularly cost-effective variant, as all film layers would only need to have a tapered end. On the other hand, the media connection or media seal would be comparatively more complex. For optimized sealing, it is also possible to compress the film stack and press a tube or a nozzle with a conical connection geometry into the media opening. The flexibility (elasticity/plasticity) of the film stack allows a sufficient media seal if the conical nozzle is pressed in with the appropriate force.

Subsequently, two further pieces of foil 96 and the corresponding intervening components, as shown in FIG. 8c, are placed on in an essentially mirror-symmetrical manner, so that the result is the application nozzle 56 shown in FIG. 6. In the embodiment example, this thus comprises a structure of five foil layers 96, 98, wherein two guide elements 86 are provided symmetrically to the central media channel 76 leading in the longitudinal direction to the treatment end 82. This is clearly recognizable in the enlarged representation of the outflow area 92 in FIG. 9.

The intended design of the application nozzle 56 and the intended manufacturing process, in particular laser cutting or punching of the contours for the media channels 76, permits enormous flexibility in the design and configuration of the cavities, cavities or media volumes provided in the layer package or laminate. Polyamide is provided as the base material for the film layers 96, 98 or film pieces 94; alternatively, however, another suitable film material such as PP or PE or even a combination of different film materials may be considered favorable. As an alternative to the use of the film pieces 94 shown in FIGS. 6 and 8, whose outer contour has already been punched out, it is also possible to carry out only the internal punching and embossing in advance and to perform the external shaping only after lamination.

In the present case, it is considered particularly important that the cleaning electrolyte is reliably applied in direct electrical contact with the anodically connected conductor elements 84 formed by the conductor wires 112. This ensures that—as intended by the concept—the current flow takes place via the electrically conductive cleaning electrolyte, which ultimately causes the desired generation of the ionic reactions and possibly the gas bubbles on the surface of the implant 2. In order to ensure this, the conductor elements 84 associated with the anodic, second electrical polarity are positioned as described in or on one of the media channels 76 in such a way that they are wetted by the cleaning electrolyte when it flows in the respective media channel 76.

An alternative embodiment of the application nozzle 56 in its structure as a film layer package is shown in a sequence analogous to FIG. 8 in FIG. 10, also starting from the first, lowermost film layer 96, with stepwise addition of the further film layers 96, 98. The variant, which is otherwise identical in construction to the embodiment according to FIG. 8, differs from the latter in the design of the conductive elements 84 provided as anode and wetted with the cleaning electrolyte during operation. In contrast to the conductive elements 84 shown in FIGS. 8c, 8d, these conductor wires 112 are designed in the embodiment example according to FIG. 10, as this becomes clear in particular from FIG. 10c, as a metal coating 120 applied to the underlying film layer 96, for example vapor-deposited (sputtered, galvanically, etc.), preferably made of gold or platinum.

A conductive element 84 formed by such a metal coating 120 has the particular advantage that it can be contoured and shaped in a particularly flexible manner. In particular, its surface contour can be adapted to the projection of the respective media channel 76 onto the foil layer 96. In this way, a particularly large contact surface, namely essentially the entire base surface of the respective media channel 76, is made available for electrical contacting of the cleaning electrolyte flowing in the media channel 76 in a material-saving manner. In particular, the distance between the conductive elements 84 and the application opening 74 can be varied and adjusted very easily as required, e.g. for further product developments or adjustments. In a particularly advantageous embodiment, which is regarded as independently inventive, the conductive element 84 is applied by a printing process, preferably a screen printing process. According to one aspect of the invention, the conductive element 84, possibly in addition to further conductive tracks, can be produced using a suitable screen printing paste, for example based on titanium. The conductor tracks preferably comprise silver, gold or titanium as the base material.

In a further aspect regarded as independently inventive, insulation or other functional structures provided between the conductive tracks may also be applied by a printing process, preferably a screen printing process. In general, furthermore, in accordance with a further independently inventive aspect, some or all of the conductor tracks may be formed from silver as the base material, whereby these may be provided with a protective coating of carbon for protection against corrosion as a result of contact with the electrolyte and/or against mechanical damage in accordance with an aspect of the invention. In particular, such silver conductor tracks can be overprinted with a carbon coating.

A further alternative embodiment of an application nozzle 56′, which is regarded as independently inventive, is shown in perspective view in FIG. 8 and in a representation of a sequence analogous to FIG. 5 in FIG. 9. In this embodiment, the application nozzle 56′ is also designed as a film layer package, the structure of which is shown in FIG. 9 by means of a sequence, also starting from the first, lowermost film layer 96. Alternatively, the conductor element 126 (preferably made of titanium, gold or platinum) is shown individually in FIG. 9b for better illustration. This conductor element 126 is firmly fixed on the underside of the foil layer 96 of FIG. 9c and is shown separately on FIG. 9b merely for reasons of understanding and illustration. In the embodiment example shown in FIGS. 8, 9, the application nozzle 56′ is designed in a manner comparable to the variant shown in FIG. 7 with anodic conductor elements 84 each formed by a metal coating.

FIG. 9a shows the lowest or first film layer 96, which is also already adapted to the desired shape of the nozzle body 72 when viewed from above. In this embodiment, the piece of film 94 already pre-cut in its outer contour in this way is provided with a central embossed groove 122. This groove 122 can also be embossed during the lamination process. In a subsequent step during the construction of the film stack, as shown in FIG. 9b, a tongue element 124 is inserted into this groove to form the cathodic conductor element 86′, the free end 104 of which projects forwards beyond the base surface of the film layer 96 formed by the piece of film 94, i.e. beyond the treatment end 82, as is also provided for the conductor element 86′ according to the design.

Analogous to the examples described above, also for the application nozzle 56′, a further foil layer 96, also a foil layer 96, is subsequently applied to the lower foil layer 96 provided in this way with the conductor element 86′ and laminated on, for example, to which the anodic conductor elements 84 are applied in the form of metal coatings 120 (FIG. 9c), analogous to the example shown in FIG. 7, and under which the tongue element 126 is fastened. The foil layer 96 in FIG. 9c is consequently coated on the underside and on the upper side with electrically conductive layers, preferably of titanium, gold and/or platinum. The next foil layer 98, formed from several pieces of foil 94, is then applied to the resulting layer package (FIG. 9d). The foil layer 98 forms the central foil layer 98, analogous to the variants described above, and is formed, at least around the circumference of one or more of the foil pieces 94 forming it, from a preferably harder foil material, i.e. in particular with a comparatively greater Shore hardness or modulus of elasticity. However, it is also possible that all film layers are made of the same material.

Also in FIG. 9d, the spaced-apart routed foil pieces 94 are recognizable, which leave openings 114 between them to form the media channels 76 integrated in the nozzle body 72. The metal coatings 120 are positioned in the two lateral media channels thus formed, which are connected in parallel on the media side, in such a way that they are wetted by the medium flowing in the respective media channel 76. In a further step, a connecting tube 120 is inserted into the receiving groove 110 of the lower film layers 96, which is also provided in this variant, to form the media connection 88 (FIG. 9d). Subsequently, as can be seen in FIG. 9d, a further film layer 96 and the corresponding intervening components, as can be seen in FIG. 9b, are also placed here in a substantially mirror-symmetrical manner. As in FIG. 9c, the film layer 96 in FIG. 9e is preferably provided on both sides with electrically conductive layers forming the two electrodes. A final film layer 96 is then applied and fixed (preferably laminated on), so that the result is the application nozzle 56′ shown in FIG. 8.

The special feature of this embodiment of an application nozzle 56′, which is regarded as independently inventive, is to be seen in the configuration of the cathodic conductor elements 86′. As mentioned above, these are fixed on a film layer 96 and form with the latter a tongue element 124, on the outside of which, i.e. in the perspective view shown in FIG. 8, a contacting electrode 126 designed as a metal coating 120 is applied on top of or below the respective projecting free end 104 projecting beyond the treatment end 82 of the nozzle body 72. Utilizing the degrees of design freedom provided by the design as a metal coating 120, this contacting electrode 126 is designed in a structured manner according to one aspect of the invention and can be designed as an element for contacting detection of the implant 2.

Such contact detection is used to recognize on the device side whether the implant 2 is also safely electrically contacted and the treatment can therefore be reliably started and carried out. In principle, several variants are conceivable for such contact detection. For example, there is the possibility of a capacitive measurement between the conductor elements 86 and the implant 2. As soon as the implant 2 is contacted by an electrode 86, the capacitance between the two conductor elements 84 and 86 changes in a measurable way.

Alternatively, a voltage can be applied inside the pocket 8 after the electrolyte has flowed through it. This results in a current flow. As soon as the implant 2 is contacted, the surface area of the electrode increases immensely, which results in an increase in current while the voltage remains constant. This can be measured and utilized.

Another possibility is to measure the voltage. As soon as the application nozzle 56, 56′ is inserted into the pocket 8 and flooded with electrolyte, a galvanic element is formed from the conductor elements 84 and 86 (anode and cathode). Preferably, the conductor elements 86 are cathodically connected and, like the implant 2 itself, are made of titanium or a titanium alloy and the conductor elements 84 are anodically connected and preferably made of gold or platinum or a metal, preferably titanium or a titanium alloy, which has been coated with gold or platinum. Due to the contacting of the implant 2, the area of the electrode that contacts it is significantly increased. This also results in a change in the voltage of the galvanic element or in the maximum possible output current. This can be measured and evaluated accordingly.

The implant contact can also be made via an impedance measurement between the two electrodes. In this case, the impedance of the electrodes also changes when the implant 2 is electrically contacted.

Another possibility is to attach one or more auxiliary electrodes to one of the aforementioned methods, which are used exclusively for implant recognition measurement with one of the measurement techniques already mentioned.

However, in a manner considered to be independently inventive, implant recognition is provided by means of the conductor element 86′ contacting the implant 2, preferably the cathode, in the embodiment shown in FIGS. 8, 9. Here, the external contacting electrode 126, as shown in an enlarged plan view in the installed state in FIG. 10 and without the last, partially insulating foil layer 96 of FIG. 10 in FIG. 11, is designed as a structured conductor element.

The metal coating 120 forming the contacting electrode 126 is divided into at least two conductor elements 128, 130 (two in the embodiment example) arranged next to each other on the tongue element 124. The conductor elements 128, 130 can be contacted separately and independently of one another. Implant detection is possible by checking for a short circuit between these conductor elements 128, 130. If the implant 2 contacts both conductor elements 128, 130 and a reliable contact is thus established between the contacting electrode 126 and the implant surface, the conductor elements 128, 130 are short-circuited via the implant 2. This can also be measured and evaluated accordingly.

In the embodiment example shown, which is regarded as particularly advantageous and independently inventive, the two conductor elements 128, 130 are designed with a comparatively complex structure, with a comparatively large number of thin conductor tracks. In the embodiment example, these are comb-shaped and arranged alternately directly next to each other. This enables a particularly effective measurement, as a short circuit can be produced in this way at a large number of local locations by implant contact. In particular, this should largely prevent the implant 2 from contacting only one of the conductor elements 128, 130. In such a case, the implant 2 would indeed be contacted and could be safely cleaned, but the electronic evaluation based on the detection of a short circuit would still not recognize any implant contact. It is therefore preferable to have alternating conductor tracks arranged next to each other, placed as close together as possible and as thin as possible. Preferably, the conductor tracks and/or the free spaces between them are narrower than 250 μm, 100 μm or 60 μm.

A further alternative embodiment of an application nozzle 56″, which is also regarded as independently inventive, is shown in perspective view in FIG. 12. In this embodiment, the application nozzle 56″ is also designed as a film layer package and, analogous to the variants described above, comprises a structure of five film layers 96, 98. In contrast to the variants described above, however, this application nozzle 56″ comprises additional application openings 132 in addition to the outflow or application openings 74 for the electrolyte arranged laterally on the nozzle body 72 in the region of the treatment end 82. According to one aspect of the invention, these are arranged on the upper and lower sides of the nozzle body 72 with respect to the planar configuration of the nozzle body 72, and thus penetrate the respective uppermost and lowermost film layer 96. The media channels 76 extending on the inside of these film layers 96 are thus also connected to the environment on the media side via the application openings 132. The implant is preferably cathodically contacted with the conductive element 86. The conductor elements 84, which are preferably anodically connected, are each located below the application openings 132 provided on both sides. The conductor elements 84 attached on both sides are not electrically connected to one another.

This embodiment is based on the concept, which is regarded as independently inventive, that in this way an automated orientation detection for the application nozzle 56″ introduced into the pocket 8 is possible. For this purpose, the metal coatings 120 arranged on both sides of the central foil 98 and forming the anodic conductor elements 84 can be electrically contacted and controlled independently of one another. Since the application nozzle 56″ is designed to be flat in its end treatment area 92, the surface of the application nozzle 56″ is usually aligned essentially parallel to the implant surface when the treatment end 82 is inserted into the pocket 8. This means that each of the conductor elements 84 is located either on the side of the central foil 98 facing the implant 2 or on the side facing away from it. Since the side facing the implant 2 has a comparatively short distance to the implant surface than the side facing away from it due to the application openings 132, it is very easy to determine which of the anodic conductor elements 84 is facing the implant 2 and which is not by means of a resistance/conductance measurement.

This can be very advantageous and desirable, as efficient and reliable energization of the implant 2 is desired and intended. On the other hand, the associated energization of the surrounding soft tissue 8 may also be undesirable and possibly lead to damage or even death of tissue parts. To avoid this, the exclusive energization and thus use of the side of the application nozzle 56″ facing the implant 2 can be provided. Since the energized anode 84 is directly and very close to the implant, in contrast to the variants in FIGS. 3 to 9, the possibly very low current flowing over the tissue is minimized to a maximum and is no longer or almost no longer present.

A still further alternative embodiment of an application nozzle 56″, which is also regarded as independently inventive, in particular with regard to its method of manufacture, is shown in a sequence of the steps of its manufacture in FIG. 16. This embodiment of an application nozzle 56″ is also based on the principle of construction as a film stack, whereby a number of functional films are used which are suitably folded over at various stages in the sense of a simplified construction method which is also suitable for large quantities.

In this variant, the media channel film 140 shown in FIG. 16a is provided as the central functional film, which will form the center film of the film layer package in the completed application nozzle 56″. Analogous to the embodiments described above, this comprises a number of pieces of film 142 which are designed and positioned in such a way that they form the media channel 76 and the application openings 74 branching off from it and connected to it. This central media channel foil 140 is embedded or wrapped in a surrounding anode foil 144, as shown in FIG. 16b. The anode foil 144, which is suitably contoured and punched out of a piece of foil, is folded around the media channel foil 140 at a fold-over point 146, so that it covers it on both sides, thereby sealingly covering the media channel 76. On the inside, and thus not visible in the representation according to FIG. 16b, the anode foil 144 is provided with a conductor track layer adapted to the “underlying” media channel 76, with which the electrical contact to the electrolyte guided in the media channel 76 is established during operation. According to an aspect of the invention, this anode conductive path layer 148 can be printed on the inside of the anode foil 144 and, according to a further aspect of the invention, comprises a suitably selected conductive material, in particular a metal such as gold or titanium. Preferably, however, and in accordance with a further aspect of the invention and in view of favorable manufacturing prices, especially for large quantities, the anode conductor track layer 148 essentially consists of silver, which in a preferred and inventive further development is provided with a carbon coating-preferably also printed on—in view of the expected contact with the electrolyte.

In order to enable the electrical contacting of the anode conductor track layers 148, which are actually located on the inside in this package design, a folding of the anode foil 144 is also provided in an independently inventive manner in the head region of the forming foil stack. In accordance with one aspect of the invention, the molded foil piece forming the anode foil 144 has two folding wings 150 in its head region (one “on top” in the folded state shown in FIG. 16b and one “on bottom” not visible in this illustration). These folding wings 150, on which the anode conductor track layer 148 is continued up to a contacting region 152, are folded over along a folding edge 154 after the anode foil 144 has been applied to the media channel foil 140 and thus come to lie on the top-side or bottom-side anode foil 144. In the illustration in FIG. 16b, this can be recognized by the side edge 156 on the anode foil 144 formed by the overlying folding wing 150. This design achieves in a simple and cost-effective manner that the contacting areas 152 come to lie on the top and bottom sides of the outside of the forming foil stack and thus become accessible for electrical contacting of the anode conductor track layer 148.

Subsequently, in a similar manner, the resulting foil package is embedded or wrapped in a cathode foil 158 surrounding it, as shown in FIG. 16c. The cathode foil 158, which is suitably contoured and punched out of a piece of foil, is folded around the package of media channel foil 140 and anode foil 144 at a fold-over point 160 and thus now forms the outermost foil layer of the now five-layer foil package on both sides. On its outer side, and thus easily accessible for electrical contacting, the cathode foil 158 is provided with a cathode conductor track layer 162. This in turn is connected to a contacting area 164. According to one aspect of the invention, the cathode foil 158 is dimensioned in such a way that, after it has been attached, its edge abuts against the side edge 156 of the folded-over folding leaf 150, so that the surface of the resulting foil packet is virtually flat.

In a further processing step, as shown in FIG. 16d, the cathode foil 158 is insulated on the outside by applying a suitable insulating material as an insulating layer 165 according to an aspect of the invention. According to one aspect of the invention, the insulating layer 165 can also be applied by a printing process, preferably a screen printing process analogous to those described above, wherein this application can take place before or after the cathode foil 158 is applied to the anode foil 144.

As a result, the nozzle body 166 shown in FIG. 16d, which can be used as an application nozzle 56″, is produced after these steps. The treatment end 168 of this nozzle body 166 is shown enlarged in FIG. 17. The layered structure of the film stack forming the nozzle body 166 resulting from the folding is clearly recognizable. The end region of the treatment end 168 is also kept free of the insulating layer 165, so that the cathode conductor track layer 162 is freely accessible in this region. The cathode conductive path layer 162, which is divided into a number of parallel conductors 170 in this area, can thus be used in the desired manner for contacting the implant.

The contact head of the nozzle body 166 shown in FIG. 16d, which can be used as an application nozzle 56″, is shown enlarged in a side view in FIG. 18. The layered structure of the film stack is also clearly recognizable here. It can also be seen that-as described above-the contacting areas 152 and 164 for the anode conductive path layer 148 and the cathode conductive path layer 162 are present both at the top and at the bottom of the foil stack, and thus in pairs, due to the foldover design used and the resulting symmetrical structure of the foil stack. Furthermore, it can be clearly seen in this embodiment that-as provided in accordance with an aspect of the invention which is regarded as independently inventive—free spaces 172 are provided in the media channel film 140 in each case between the pairs of contacting regions 152, 164, which allow elastic deformation of the stack towards the inside in the region of the contacting regions 152, 164 due to the elastic properties of the films. The entrance 174 of the media channel 76, via which it can be connected to a suitable media reservoir, is also recognizable in this embodiment.

According to one aspect of the invention, the application nozzle 56″ comprising the nozzle body 166 can be used to provide a treatment head 180 by providing it with a suitable housing 182 in the connection area. The treatment head 180 thus formed, which is regarded as independently inventive, is shown in partial section in FIG. 19 and in side view in FIG. 20. In a manner considered to be independently inventive, the nozzle body 166 is thereby guided into the housing 182 by means of a suitable overmolding of elastic material, preferably a rubber-silicone overmolding. Similarly, the fluid inlet, i.e. the connection of the media inlet 174 with a corresponding media reservoir, can be made of elastic material or with elastic overmolding. Thus, in addition to the high tightness of the system, which is favorable for use, the correct alignment of the nozzle body 166 itself and with respect to the handpiece is also possible in a particularly simple manner, as is the sealing of the supply hose, the sealing to the handpiece including the connection contacts, so that it is possible to dispense with additional sealing elements such as O-rings.

In an embodiment which is regarded as independently inventive, the treatment end 168 of the nozzle body 166 can be designed for an automatable contact detection with the component to be treated, in particular the dental implant 2, by means of suitable conductor routing of the cathode conductor track layer 162. In this embodiment, which is regarded as independently inventive, the conductors 170 of the cathode conductor track layer 162 in the region of the treatment end 168′, i.e. in the region of the transfer point 160, are not designed to be in continuous contact with one another, but are subdivided into two conductor groups which interlock in a comb-like manner. The conductors 170a of the first conductor group, which are arranged alternately to the conductors 170b of the second conductor group in the area of the transfer point 160, are connected exclusively to one of the external contacting areas 164, and the conductors 170b of the second conductor group are connected exclusively to the other contacting area 164. In the “normal” state, there is thus no electrically conductive connection between the two contacting areas 164, and the detection of a mechanical and/or electrical contact with the component 2 to be treated can be carried out on the basis of the detection of an electrically conductive connection between the two contacting areas 164.

For the electrical contacting of the application nozzle 56″, the targeted use of the deformability of the foil stack of the nozzle body 166 is provided in accordance with an aspect that is regarded as independently inventive. This is based on the realization that a resilient element is usually provided for electrical plug connections, with which the electrical contact is reliably established after a mechanical contact has been made using the spring force of such an element. In order to utilize this for the nozzle body 166 in a particularly reliable and simple manner, the use of the spring force of the foil stack of the nozzle body 166, which is in itself already fundamentally elastic, is provided. For this purpose, the above-mentioned free spaces 172 between the respective pairs of contacting areas 152, 164 are provided in an inventive manner. To establish the electrical connection in the sense of a plug connection, a contact plug 190 is provided, which is shown in perspective in front view in FIG. 22 and in rear view in FIG. 23. According to one aspect of the invention, the contact plug 190 comprises a front contact area 192, which is provided for establishing a secure mechanical and electrical contact with the treatment head 180, and a rear contact area 194, which can be connected to corresponding further systems.

In the frontal contact area 192, the contact plug 190 is provided on the one hand for making reliable mechanical contact with the treatment head 180. It therefore comprises jaw-like housing halves 196, which are arranged opposite one another to form a clear gap 198. The clear gap 198 is dimensioned in such a way that, within the manufacturing tolerances, it corresponds approximately slightly less than the total height of the film stack forming the nozzle body 166. The aim of the design is that the film stack should be able to be inserted into the clear gap 198 without excessive mechanical stress, whereby it should be slightly crushed and thus fixed. This basically enables a mechanically reliable attachment of the nozzle body 166 in the frontal contact area 192 of the contact plug.

In addition, the contact plug 190 comprises two pairs of electrical contact plugs 200, 202 in its front contact area 192, which are each electrically connected to associated rear contact plugs 204, 206. The front contact plugs 200, 202 are each provided in pairs for making electrical contact with the contacting areas 152, 164. In order to establish a reliable electrical contact, the use of the elasticity of the foils of the foil stack forming the nozzle body 166 in combination with the aforementioned free spaces 172 is provided in an embodiment which is regarded as independently inventive. The free spaces 172 namely allow a retreating deformation of anode foil 144 and cathode foil 158 in this spatial region into the free spaces 172. Accordingly, according to this aspect of the invention, the contact plugs 200, 202 of each pair are spaced apart less than the total thickness of the foil stack and thus also the clear width of the clear gap 198. By inserting the foil stack into the frontal contact area 192, the contact plugs 200, 202 are thus each pushed onto an associated contact area 152, 164 in a manner considered to be inventive, whereby the latter recedes resiliently into the corresponding free space 172 due to the dimensioning.

This design makes it possible to realize the normally provided spring-loaded contacting of an electrical contact within the nozzle body, which is designed as a disposable product, so that the reliability, service life and wear of the other system components can be kept correspondingly low.

### List of Reference Symbols

1, 1′ Treatment system
2 Dental implant
4 External thread
6 Jaw bone
8 Pocket
10 Soft tissue
12 Handle
14 Battery
16 Storage container
18 Transition piece
20 Treatment head
22, 24 Supply lines
26 Connection side
28 Treatment end
30 Base body
32 Media channel
34, 36 Conductor element
38 Emission range
40 Outlet opening
52 Supply unit
54 Handle
56, 56′, 56″,
56′″ application nozzle
57 PVC or silicone hoses
58 Electrolyte ampoule
60 Connecting cable
62 Control unit
64 Buffer battery
66 Pump
67 Housing
68 Hose valve
70 Pressure chamber
72 Nozzle body
74 Application opening
76 Media channel
78 Electrical conductor element
80 Connection area
82 Treatment end
84, 86 Conductor element
88 Media connection
90 Branching point
92 Emission range
94 Foil piece
96, 98 Foil layer
100 Spacer
102 Nut
104 End
106 Conducting wire
107 Contact opening
108 Nut
110 Recording groove
112 conductor wire
114 Recess
116 End segment
118 Contact holes
120 Metal coating
122 Nut
124 Tongue element
126 Contact electrode
128, 130 Conductor element
132 Application openings
140 Media channel film
142 Foil piece
144 Anode foil
146 fold-over point
148 Anode track layer
150 Folding sash
152 Contact area
154 Folding edge
156 Lateral edge
158 Cathode foil
160 fold-over point
162 Cathode track layer
164 Contact area
165 Insulating layer
166 Nozzle body
168, 168′ Treatment end
170, 170a,
170b Conductor
172 Free space
174 Input
180 Treatment head
182 Housing
190 Contact plug
192 frontal contact area
194 Rear contact area
196 Housing halves
198 light gap
200, 202 electrical contact plugs
204, 206 rear contact plug
d Foil thickness
D Total thickness

1. An application nozzle for applying a dental active substance in the oral cavity of a patient, in particular for a system for cleaning an implant part contaminated with biofilm, having a base or nozzle body in which, on the one hand, at least one media channel and, on the other hand, a number of electrical conductor elements are integrated, the base or nozzle body being designed as a base or nozzle body which extends flat in a longitudinal direction from a connection region towards a free treatment end and tapers in its cross-section in the direction of the treatment end.

2. An application nozzle according to claim 1, in which the conductor element or elements are positioned in or on one of the media channels in such a way that they are wetted by the cleaning electrolyte as it flows in the respective media channel.

3. An application nozzle according to claim 1, in which at least one of the media channels branches out within the base or nozzle body and opens on the outlet side into a plurality of application openings arranged in an outflow region provided in the region of the treatment end.

4. An application nozzle according to claim 1, in the base or nozzle body of which at least two conductor elements assigned to a common first electrical polarity are integrated.

5. An application nozzle according to claim 4, in which the conductor elements assigned to the first electrical polarity are positioned in or on one of the media channels in such a way that they are wetted by the cleaning electrolyte supplied from a common electrolyte ampoule as it flows in the media channels.

6. An application nozzle according to claim 1, in which a conductor element assigned to a second electrical polarity protrudes beyond the treatment end formed by the base or nozzle body as viewed in the longitudinal direction.

7. An application nozzle according to claim 1, in which a number of the application openings are arranged in an out-flow direction aligned laterally to the longitudinal direction.

8. An application nozzle according to claim 1, the base or nozzle body of which has a contour shaped in the manner of a triangle in plan view.

9. An application nozzle according to claim 1, the nozzle body of which is designed as a laminate of a plurality of pieces of film.

10. Application nozzle according to claim 9, in which the media channels in a film layer of the laminate are formed by an inserted recess in the respective layer film.

11. An application nozzle according to claim 9, the media channels of which are provided with integrated spacers.

12. An application nozzle according to claim 9, the nozzle body of which is constructed from at least three film layers, wherein a number of film pieces forming a central film layer arranged between two adjacent film layers are formed from a harder film material than the two adjacent film layers.

13. An application nozzle according to claim 1, which is designed as a disposable product.

14. A System for cleaning a component contaminated with biofilm, in particular an implant part, with an application nozzle according to claim 1, and with a handle which is provided with a number of electrical and media connections in such a way that both the electrical conductor elements and the media channels of the application nozzle can be connected to corresponding electrical or media supply lines in the handle or in an associated transition piece, media supply lines in the handle or in an associated transition piece.

15. A System according to claim 14, in the handle of which a replaceable reservoir for cleaning electrolyte is arranged.
